(19) United States
(12) Patent Application Publication
(10) Pub. No.: US 2008/0306694 A1
(43) Pub. Date: Dec. 11, 2008

(54) Methods for Detecting Peaks in a Nucleic Acid Data Trace

(75) Inventors: Alexandre M. Izmailov, Etobicoke (CA); Murugathas Yuwaraj, Markham (CA)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS HEALTHCARE DIAGNOSTICS INC., Tarrytown, NY (US)

(21) Appl. No.: 12/158,766

(22) PCT Filed: Feb. 6, 2007
(86) PCT No.: PCT/US2007/061698

§ 371 Date: Jun. 23, 2008

Related U.S. Application Data
(60) Provisional application No. 60/765,507, filed on Feb. 6, 2006.

(51) Int. Cl. C12Q 1/68; G06F 19/00

(52) U.S. Cl. 702/19

(57) ABSTRACT

The present invention relates to methods and apparatus for detecting peaks in a sample nucleic acid data trace derived from a sample polynucleotide by (a) receiving a sequence signature of a reference polynucleotide, wherein the sequence signature comprises a profile of peak height at one or more peak position of a nucleic acid sequence data trace of one or more of reference polynucleotides; (b) receiving a sample nucleic acid sequence data trace of a sample polynucleotide corresponding to the reference polynucleotide, wherein the sample nucleic acid sequence data trace comprises a value of peak height at one or more peak position corresponding to the peak positions of the sequence signature; and (c) detecting peaks in the sample nucleic acid data trace having a peak height that correlates with the profile of peak height of the sequence signature at a corresponding peak position.

### CROSS REFERENCE TO RELATED APPLICATION

This application claims the benefit of the filing date of U.S. Provisional Patent Application No. 60/765,507, filed Feb. 6, 2006, the disclosure of which is incorporated, in its entirety, by this reference.

### FIELD OF THE INVENTION

The present invention relates generally to the field of analysis of chromatographic signals representing separation patterns of mixtures of molecules, such as nucleic acid sequences.

### BACKGROUND OF THE INVENTION

Mixtures of molecular compounds are often separated into their various constituents using chromatographic techniques, based upon their differential migration or movement through a sieving medium according to certain properties, such as molecular weight, or affinity for a solid adsorbent. The separated constituent compounds may be visualized by a number of different techniques, most of which require that the constituent compounds be labeled with a molecule that emits electromagnetic radiation, such as a fluorescent dye. The radiation emitted by the labeled molecule can be detected by an optical detector sensitive in the spectral range of emitted radiation and then converted to an electronic or visual signal indicating the identity, amount, and order of the labeled fragments.

Chromatographic methods are commonly used to determine the sequence of a nucleic acid sample. Such methods involve the electrophoretic separation of mixtures of nucleic acid chain-termination fragments representing a size-distribution of fragments terminating at each A, G, T and C of the nucleic acid, with each fragment being labeled with a detectable label specific to the base type (A, T, G, or C) of the last nucleotide base of the fragment (in the case of dye-terminator labeling chemistry). Alternatively, the primer used in the sequencing reaction can be labeled. The chain termination fragments are electrophoretically separated in a gel medium according to the fragment size, resulting in a pattern of bands corresponding to the order of the terminal nucleic acid base type. An optical detector detects the signal emitted by the fragment labels in the order of migration and converts the signal to a visualized pattern of peaks representing discrete constituent terminal nucleotide bases of each fragment. The pattern of peaks can then be analyzed by signal processing technology and/or computer, to determine the order, quantity, and identity of the terminating base type (and hence the sequence) of the individual components nucleic acid sample.

Because chromatographic methods of nucleic acid sequencing utilize an electrophoretic sieving medium to separate DNA fragments on the basis of size, the accuracy of the sequence results depends on accurate detection of the chronological order in which the fragments migrate through the medium, as indicated by the presence and order of signal peaks representing individual fragments in an chromatogram or sequence data trace. Failure to identify a peak will result in loss of a base (called deletion error) in the identified sequence where a base actually exists. Identification of a false-positive peak (a peak that does not in fact represent a real nucleotide fragment) will result in a nucleotide/base being inserted (insertion error) in the identified sequence where no base actually exists.

Accurate identification of the order, identity and quantity of constituent components (e.g., chain termination fragments terminating with and indicative of particular nucleic acid base types) of a chromatographic separation process is critical for many applications. However, the accuracy of current methods is limited by a number of factors. For example, identification of nucleotide sequences using traces obtained during electrophoresis process (in a slab gel or capillary-based system) is severely limited by the large variation in the yield of the amplified products. This variation in yield is manifested as large variations in the heights of peaks present in the corresponding chromatogram. The large variation in peak height is compounded when fluorophores (dyes) are used to tag the terminating nucleotide in a DNA fragment (called dye-terminator chemistry). As a result in variations inherent in dye-terminator chemistry, the general intensity of labeling often varies between the four nucleotide types. There is also a tendency for signals of a given type of nucleotide to vary unpredictably in relative intensity. Conventional methods often assume a gradual variation in peak heights over the course of the sequence and attempt to identify peaks based on this assumption. This model breaks down, however, when large variations are present in the peak heights. When large variation in peak heights results in some peaks with heights as low as the noise level in the data, such peaks cannot be clearly distinguished from noise peaks. Hence, bases corresponding to such small peaks are omitted in the identified sequence (false negatives). Such errors are known as deletion errors. The above factors contribute to difficulties in accurately identifying individual constituent peaks, ordering of the peaks, and determining the correct sequence of bases.

Various methods have been utilized to circumvent the above problems and improve the accuracy of base-calling, including highly configurable data processing modules, homomorphic deconvolution followed by peak detection, neural networks, grid search assuming regularly spaced Gaussian pulses, expert systems, and others. The various methods generally fall into two categories: deconvolution methods and peak-fitting methods. Deconvolution methods, on the one hand, are based on an unbiased interpretation of data inherent in the peak data generated by the sample sequence, and involve an enhancement of the data by means of computational elimination or reduction of variables contributing to the blurring of the peak, which should theoretically results in an ideal discrete profile peak. Typical deconvolution base-calling methods use simple Fourier methods to predict base positions and then find peaks in the data as regions about inflexions or concavities in the signal that exceed certain area thresholds. Deconvolution methods, however, have limited utility where such inflexions between peaks are not present. Deconvolution is also highly sensitive to noise. Peak-fitting methods, on the other hand, are based on empirical knowledge of the number, location, and characteristics of peaks of the same or a cognate sequence. Most published and known methods of peak detection and sequence identification from chromatograms employ a peak height model that represents a smoothed (and averaged) profile of peak heights that is expected to vary only gradually from the start of the sequencing run to the end. See, for example, Tibbetts et al., U.S. Pat. No. 5,365,455, and Tibbetts et al., Neural Networks for Automated Base-calling of Gel-based DNA Sequencing Ladders, Automated DNA Sequencing and Analysis, Chapter 31.

There is a continuing need for improved methods of base-calling, particularly for methods that are capable of accurately detecting peaks in chromatograms containing large variations in peak characteristics, such as peak height.

### SUMMARY OF THE INVENTION

The present invention relates to methods for detecting peaks in a nucleotide sequence signature of a sample polynucleotide, which is capable of accounting for large variation in peak characteristics, such as peak height, by utilizing a profile of the peak characteristics generated empirically from other samples of the polynucleotide. Instead of assuming a gradual variation in peak heights, the method of the present invention uses the relative variation, or the range of variation, of peak height within a chromatogram as a “signature” or “profile” that is conserved for a given sequence. Because relative peak heights are expected to be conserved between sequencing runs of the same sample and remain fairly consistent between samples when the sequences are conserved, this empirical information can be effectively used in cases where a majority of the DNA sequence is known to remain constant. Hence, sequence analysis can be done by utilizing high-level contextual information, such as knowledge of the conserved sequence and regions where mutations are expected to occur. One advantage of the present invention is that the method and apparatus is able to capture and utilize information that is conserved among sequences of multiple samples, without loss of vital information. The present invention is therefore able to account for any variation in peak characteristics, such as peak height, where the profile is repeatable across different runs. Accordingly, the methods of the invention utilize empirical knowledge of the sequence to improve the accuracy with which variations in characteristics of the known sequence can be identified.

The methods of the present invention are useful, for example, in accurately determining the sequence of a sample polynucleotide that is associated with a disease or condition among a population of subjects, and that is therefore the object of repeated detection and analysis. The ubiquitous presence of such a polynucleotide enables use of empirical information from one sample to be used as a reference standard in determining the sequence of other samples. The methods of the present invention improve accuracy of sequencing DNA known to remain substantially unchanged, for example, DNA-based resistance testing in HIV, where approximately 90% of the viral sequence is known to remain unchanged.

The sequence profile of the present invention can be utilized in various ways in accordance with the invention to accurately detect peaks in a new chromatogram obtained from a similar (but not necessarily identical) DNA sample.

One aspect of the present invention relates to methods for detecting peaks in a sample nucleic acid data trace derived from a sample polynucleotide. In one embodiment, the method comprises: receiving a sequence signature of a reference polynucleotide, wherein the sequence signature comprises a profile of peak height at one or more peak position of a nucleic acid sequence data trace of one or more reference polynucleotides; receiving a sample nucleic acid sequence data trace of a sample polynucleotide corresponding to the reference polynucleotide, wherein the sample nucleic acid sequence data trace comprises a value of peak height at one or more peak position corresponding to the peak positions of the sequence signature; and detecting peaks in the sample nucleic acid data trace having a peak height that correlates with the profile of peak height of the sequence signature at a corresponding peak position.

In another embodiment of the above method, the sequence signature of the reference polynucleotide comprises a profile of peak height of peaks of one or more nucleic acid bases as a function of peak position of a nucleic acid sequence data trace of a single reference polynucleotide.

The methods of the present invention may utilize the sequence signature in any one of various ways. For example, the step of detecting peaks in the sample nucleic acid data trace may comprise normalizing peak heights of the sample nucleic acid sequence data trace and detecting peaks in the sample nucleic acid sequence data trace having approximately uniform height. Peak heights of the sample nucleic acid sequence data trace may, for example, be normalized by the inverse of the peak height of a corresponding peak of the sequence signature.

In another embodiment, the step of detecting peaks in the sample nucleic acid data trace may comprise detecting peaks in the sample nucleic acid data trace having a peak height approximately equal to the peak height of a corresponding peak of the sequence signature.

In yet another embodiment, the step of detecting peaks in the sample nucleic acid data trace may comprise determining for each peak position of the reference polynucleotide a value encompassing the variance in peak height from an average peak height of a plurality of nucleic acid bases of the reference polynucleotide, and detecting peaks in the sample nucleic acid data trace at a corresponding base position having a peak height within said variance.

Another aspect of the present invention relates to an apparatus for detecting peaks in a sample nucleic acid data trace derived from a sample polynucleotide. In one embodiment, the apparatus comprises:

(a) an input to receive information relating to a nucleic acid sequence data trace of one or more reference polynucleotides corresponding to the sample polynucleotide and to receive information relating to a nucleic acid sequence data trace of a sample polynucleotide;

(b) one or more processor operatively programmed to collectively perform the following processes:
evaluate the nucleic acid sequence data trace of the one or more reference polynucleotides and generate a sequence signature comprising a profile of peak height, as a function of peak position, at one or more peak position of the nucleic acid sequence data trace of the one or more reference polynucleotide;
evaluate the sample nucleic acid sequence data trace and generate a value of peak height, as a function of peak position, at one or more peak position corresponding to a peak position of the sequence signature;
detect peaks in the sample nucleic acid data trace having a peak height that correlates with the profile of peak height of the sequence signature at a corresponding peak position, thereby identifying valid peaks in the nucleic acid sequence data trace of the sample polynucleotide; and

(c) an output to report detected peaks in the nucleic acid sequence data trace of the sample polynucleotide.

In another embodiment, the present invention relates to a computer system for detecting peaks in a sample nucleic acid data trace derived from a sample polynucleotide, comprising:

(a) an input to receive information relating to a nucleic acid sequence data trace of a reference polynucleotide corresponding to the sample polynucleotide and to receive information relating to a nucleic acid sequence data trace of a sample polynucleotide;

(b) one or more processor operatively programmed to collectively perform the following processes:
evaluate the nucleic acid sequence data trace of the reference polynucleotide and generate a sequence signature comprising a profile of peak height, as a function of peak position, at one or more peak position of the nucleic acid sequence data trace of the reference polynucleotide;
evaluate the sample nucleic acid sequence data trace and generate a value of peak height, as a function of peak position, at one or more peak position corresponding to a peak position of the sequence signature;
detect peaks in the sample nucleic acid data trace having a peak height that correlates with the profile of peak height of the sequence signature at a corresponding peak position, thereby identifying valid peaks in the nucleic acid sequence data trace of the sample polynucleotide; and

(c) an output to report detected peaks in the nucleic acid sequence data trace of the sample polynucleotide.

The apparatus or computer system of the present invention may utilize the sequence signature in any one of various ways. For example, in one embodiment, the processor is programmed to detect peaks in the sample nucleic acid data trace by normalizing peak heights of the sample nucleic acid sequence data trace and detecting peaks in the sample nucleic acid sequence data trace having approximately uniform height. Peak heights of the sample nucleic acid sequence data trace may, for example, be normalized by the inverse of the peak height of a corresponding peak of the sequence signature.

In another embodiment, the processor is programmed to detect peaks in the sample nucleic acid data trace having a peak height approximately equal to the peak height of a corresponding peak of the sequence signature.

In yet another embodiment, the processor is programmed to determine, for each peak position of the reference polynucleotide, a value encompassing the variance in peak height from an average peak height of a plurality of nucleic acid bases of the reference polynucleotide, and to detect peaks in the sample nucleic acid data trace at a corresponding base position having a peak height within said variance.

In another aspect, the present invention includes a computer system for detecting peaks in a sample nucleic acid data trace derived from a sample polynucleotide that performs the processes described above.

In another aspect, the present invention includes a computer readable medium having stored thereon computer executable instructions for performing methods for detecting peaks in a sample nucleic acid data trace derived from a sample polynucleotide that performs the processes described above.

These and other embodiments of the invention are described in more detail below.

### BRIEF DESCRIPTION OF FIGURES

FIG. 1 is a diagram illustrating various possible data analysis methods to accurately detect peaks on the basis of a peak height profiles.

FIG. 2 is an illustration showing one possible method of generating a peak profile by normalization of peak heights using the inverse of the signature, resulting in peaks heights that are closer to a uniform value, and enabling use of simple peak detection to identify base sequences. The arrows indicate the expected transformation of peak heights towards a uniform value.

FIG. 3 is an illustration showing the use of peak height profile as a model in peak detection. If peaks heights are assumed to vary only gradually according to the “classical average model” (dotted line), then small peaks (such as peak 4) fall outside the scope of the acceptance criteria and will be omitted as outliers. A peak height profile model (solid curve), establishes acceptance criteria that captures (detects) peaks that are consistently small.

FIG. 4 is an illustration showing the use of the peak profile signature to modify acceptance criteria. In this method, peak height profile is used to adjust the acceptance criteria according to the degree of variation empirically observed. This method is analogous to the method illustrated in FIG. 4, except that the classical average model is assumed to hold true, while the window within which the peak height is allowed to vary is modified according to the peak height profile (signature). Hence, the window is broad at positions where small peaks consistently occur (e.g., peak 4) and narrow at positions where peaks are consistently close to the “classical average model” (dotted line) (e.g., peak 1).

FIG. 5 is a plot showing a high correlation between respective peak heights in two different machine runs sequencing the same sample.

FIG. 6 is a plot of scaled raw peak heights (open dots) and adjusted HIV peak heights (filled dots), showing that use of correlation information in accordance with the methods of the present invention significantly reduce peak height variability.

FIG. 7 shows sequence data traces resulting from use of the methods of the present invention to base-calling of the M13 sequence. FIG. 7a is a sequence data trace, comparing a raw data trace having an obscured peak with a data trace showing the obscured peak after compensation of peak heights, using a profile generated based on a linear model. Similarly, FIG. 7b shows that the obscured peak in the raw data trace can be detected using a tolerance window that tracks the actual height profile as a functio of peak position. FIG. 7c also shows that the obscured peak in the raw data trace can be detected using a tolerance window that reflects the value of deviation of the profile from an average value (the average value representing the traditional linear model).

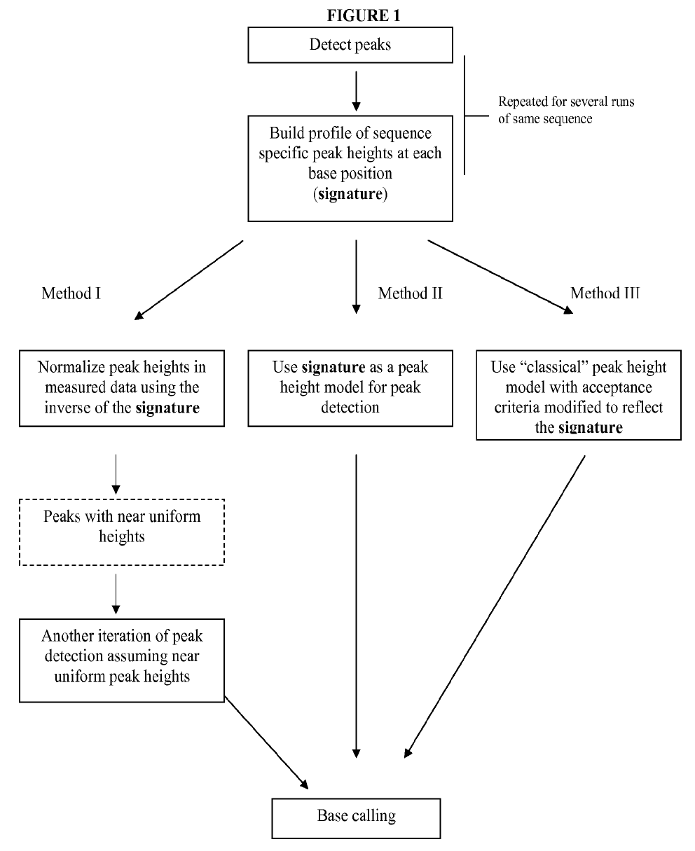

### DETAILED DESCRIPTION OF THE INVENTION

The present invention generally provides a method for detecting peaks in a data trace of a nucleotide sequence signature used for base-calling. In accordance with the invention, the method utilizes empirically observed conserved peak characteristics, such as peak height, derived from a reference sequence, as a “signature” or “profile” to modify acceptance criteria for detecting peaks in a data trace. By adjusting the acceptance criteria for valid peaks to allow for large deviations in conserved peak characteristics, peaks may be detected with a higher degree of accuracy and repeatability.

In the following description, numerous specific details of programming, software modules, user options, networks, database queries, database structures, etc., are provided for an understanding of various embodiments of the systems and methods disclosed herein. However, those skilled in the art will recognize that the systems and methods disclosed can be practiced without one or more of the specific details, or with other methods, components, materials, etc.

In some cases, well-known structures, materials, or operations are not shown or described in detail. Furthermore, the described features, structures, or characteristics may be combined in any suitable manner in one or more embodiments. It will also be readily understood that the components of the embodiments could be arranged and designed in a wide variety of different configurations.

The order of the steps or actions of the methods described in connection with the embodiments disclosed may be changed as would be apparent to those skilled in the art. Thus, any order in the figures or detailed description is for illustrative purposes only and is not meant to imply a required order.

Several aspects of the embodiments described may be implemented as software modules or components. As used herein, a software module or component may include any type of computer instruction or computer executable code located within a memory device and/or transmitted as electronic signals over a system bus or wired or wireless network. A software module may, for instance, comprise one or more physical or logical blocks of computer instructions, which may be organized as a routine, program, object, component, data structure, etc., that performs one or more tasks or implements particular abstract data types.

In certain embodiments, a particular software module may comprise disparate instructions stored in different locations of a memory device, which together implement the described functionality of the module. Indeed, a module may comprise a single instruction, or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices.

Software modules or instructions may be carried out, for instance, on a computer having a processor that communicates with the one or more memory devices listed above having stored thereon the software modules or instructions. The computer may be a personal computer, a server, a laptop, a handheld device, or another processing device known in the art.

### DEFINITIONS

While the terminology used in this application is standard within the art, the following definitions of certain terms are provided to assure clarity.

Units, prefixes, and symbols may be denoted in their SI accepted form. Unless otherwise indicated, nucleic acids are written left to right in 5′ to 3′ orientation. Numeric ranges recited herein are inclusive of the numbers defining the range and include and are supportive of each integer within the defined range. Unless otherwise noted, the terms “a” or “an” are to be construed as meaning “at least one of.” The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. In the case of any amino acid or nucleic sequence discrepancy within the application, the figures control.

The terms “nucleic acid” and “polynucleotide” are considered to be equivalent and interchangeable, and refer to polymers of nucleic acid bases comprising any of a group of complex compounds composed of purines, pyrimidines, carbohydrates, and phosphoric acid. Nucleic acids are commonly in the form of DNA or RNA. The term “nucleic acid” includes polynucleotides of genomic DNA or RNA, cDNA, semisynthetic, or synthetic origin. Nucleic acids may also substitute standard nucleotide bases with nucleotide isoform analogs, including, but not limited to iso-C and iso-G bases, which may hybridize more or less permissibly than standard bases, and which will preferentially hybridize with complementary isoform analog bases. Many such isoform bases are well-known in the art. The nucleotides adenosine, cytosine, guanine and thymine are represented by their one-letter codes A, C, G, and T respectively. In representations of degenerate primers or mixtures of different strands having mutations in one or several positions, the symbol R refers to either G or A, the symbol Y refers to either T/U or C, the symbol M refers to either A or C, the symbol K refers to either G or T/U, the symbol S refers to G or C, the symbol W refers to either A or T/U, the symbol B refers to “not A”, the symbol D refers to “not C”, the symbol H refers to “not G”, the symbol V refers to “not T/U” and the symbol N refers to any nucleotide.

The term “polynucleotide” may refer to either a particular polynucleotide sample, or alternatively to the polynucleotide sequence at a particular genetic locus. For example, when referring to a polynucleotide sequence of the gp41 region of HIV-1, it is understood that HIV-1 exists in nature as multiple species and quasi-species, and that the polynucleotide sequence of the gp41 region may vary from species to species. The term “gp41 polynucleotide” may therefore refer generally to the polynucleotide sequence of the gp41 region, which is found in various species of HIV-1 but has a common or conserved sequence with limited variation in the exact sequence, and may be isolated from different individuals, or found in different samples of the same individual who may have been infected with different species of HIV-1. Alternatively, the term “gp41 polynucleotide” may refer to a specific polynucleotide sequence of a gp41 region of an individual species of HIV-1 from a single sample. As used in the claims describing the present invention, the term “polynucleotide” is used in reference to a “sample polynucleotide,” as well as to “reference polynucleotides.” The term “sample polynucleotide” means the polynucleotide of a single sample. Typically, a sample polynucleotide will be derived from a single source, such as a single individual, a single tissue, or a single cell. The sample polynucleotide is a particular polynucleotide, whose sequence is being determined. The term “reference polynucleotides,” on the other hand, means polynucleotides other than the sample nucleotide. The reference polynucleotides are polynucleotides whose sequences are used as a reference standard against which the sample polynucleotide sequence is compared. Although the reference polynucleotides will not be the same physical polynucleotide as the sample nucleotide, the reference nucleotides may be isolated from either the same individual, tissue, or cellular source (and may therefore actually have the identical nucleotide sequence as the sample polynucleotide), or may be isolated from an entirely different source, such as a different individual (and may therefore have the identical nucleotide sequences as the sample polynucleotide, or may have a nucleotide sequence that differs to varying degrees).

The term “data trace” refers to a graphical or numerical representation of a signal, typically in the form of a series of peaks and valleys, representing a chromatographic separation of set of nucleic acid chain termination fragments produced in a chain termination sequencing reaction for a specific nucleic acid sequence and detected in a DNA sequencer. Data traces are also sometimes referred to as a “chromatogram” or “sequence signature.” A data trace is produced by a system that detects a plurality of discrete molecular entities separated from a mixture into their various constituents by differential migration or movement through a sieving medium on the basis of different physical properties, such as molecular weight or affinity for a solid adsorbent. A data trace is generally an array of numbers, typically represented as a plot corresponding to a signal generated by a source of electromagnetic radiation emissions versus time or relative position of components migrating within the mobile phase. In the context of nucleic acid sequencing, a data trace is generated as a result of the electrophoretic separation of nucleic acid fragments of different size over time. The data trace usually comprises one or more peaks, each peak representing the location of an individual component relative to other components (plotted horizontally on the time or molecular weight scale), with the area under the peak providing a quantitative measure of that component (plotted vertically on the signal intensity scale). Although a data trace is generally in the form of a graphical representation of peaks and valleys, it is to be understood that a data trace may also take the form of a stream (array) of numerical values or, alternatively, a mathematical (i.e., polynomial) expression or function, or a database of values (i.e., peak characteristics, such as peak height, peak width, peak area, etc.) as a function of time or relative position in the order of migration. The data traces used in the present invention may be either a raw data trace or a conditioned data trace that has undergone some preliminary processing.

The term “peak” means a detectable extremity representing a signal obtained from an electromagnetic radiation emission associated with one or more components of a mixture separated in an electrophoretic medium. A peak is graphically represented on a chromatogram approximately as a bell-shaped function having one or more a values that represent characteristics or properties of each electromagnetic signal received from discrete molecular entities in the separation medium. Peaks are generally represented as a series of signals measured at selected intervals of time or space by a digitizing scanner to detect, for example, (i) electromagnetic radiation emanating from a distinct components separated on an electrophoretic gel, (ii) electromagnetic radiation emanating from distinct components separated within a capillary gel electrophoresis device, or (iii) the optical density of an image on an exposed film, such as an autoradiograph, representing electromagnetic radiation from an electrophoretic gel. The term “peak,” as used herein, refers to both “well-defined peaks” (see definition below), which is in essentially all instances equivalent to a “constituent peak” (see definition below), as well as to “composite peaks” in low-resolution regions of a chromatogram that consist of two or more constituent peaks that cannot be resolved. The term “peak,” as used in the context of a peak of a “reference polynucleotide,” also refers to peaks representing a combination of peak characteristics derived from a plurality of polynucleotides (i.e., peaks that do not represent a single physical constituent, but rather a set of multiple components or constituents, blended or combined in such a way that the “peak” represents an average value or a range of values representative of the set as a whole).

The term “constituent peak” means a peak representing a single component of a mixture of components.

The term “sample” means a compound or mixture of compounds that is the subject of analysis. A sample polynucleotide is a polynucleotide whose polynucleotide sequence is being determined.

The term “sequence signature” refers to a chromatographic signal representing a distribution of nucleic acid chain-termination fragments of the specific nucleic acid sequence.

The term “peak height” means the maximum amplitude of a peak.

The term “peak spacing” or “inter-peak spacing” means distance between two successive or adjacent peaks in a chromatogram.

The term “peak area” means the total area under a curve defining a peak.

The term “peak width” means the full width measured at half of the maximum amplitude of the peak.

The term “peak resolution” means the ratio of peak spacing and peak-width.

The term “profile” means a function in which the values of one variable correspond to the values of another variable. The term “profile” encompasses the correspondence or association of empirically determined values, as well as the correspondence or association of values extrapolated from empirically determined values. A model will generally take the form of a curve fitted to empirically determined data, which curve may be represented graphically, numerically, or mathematically in the form of a polynomial function. In the context of the present invention, a profile of multiple peaks represents a consensus value, or alternatively a range of values, representing peak heights of a plurality of nucleotides representing a given sequence. The profile may be represented, for example, as a set of single values of peak heights, a set of average values of peak heights, or a set of values representing a range of peak height values.

The term “reference sequence” means a nucleotide sequence of a polynucleotide corresponding to the same genetic sequence as the sample sequence. Where the likely sequence of the DNA being analyzed is known, for example in repetitive diagnostic applications of a particular genetic sequence, the known nucleotide sequence may be used to generate a set of model data traces used as a reference sequence, which is compared with the nucleotide sequence obtained from the sample.

The term “sample sequence” means a nucleotide sequence of a polynucleotide corresponding to a target polynucleotide present in the sample that is the object of diagnostic inquiry.

The term “sequencing” means the chemical process of generating fragments of nucleic acid or polynucleotide molecules in order to determine the identity and order of nucleotides in a molecule. A well known method of sequencing is the “chain termination” method first described by Sanger et al., PNAS (USA) 74(12): 5463-5467 (1977) and detailed in Sequenase® 2.0 product literature (Amersham Life Sciences, Cleveland) and more recently elaborated in European Patent EP-B1-655506, the content of which are all incorporated herein by reference. In this process, DNA to be sequenced is isolated, rendered single stranded, and placed into four vessels. In each vessel are the necessary components to replicate the DNA strand, which include a template-dependent DNA polymerase, a short primer molecule complementary to the initiation site of sequencing of the DNA to be sequenced and deoxyribonucleotide triphosphates for each of the bases A, C, G and T, in a buffer conducive to hybridization between the primer and the DNA to be sequenced and chain extension of the hybridized primer. When a primer hybridizes to each strand of DNA, the DNA polymerase initiates synthesis (extension) of a new strand of DNA by adding one base at a time that is complementary to the corresponding base of the template strand, to form a new nucleic acid polymer complementary to the template DNA. Each vessel also contains a small quantity of one type of “chain-terminating” dideoxynucleotide triphosphate, e.g. dideoxyadenosine triphosphate (“ddA”), dideoxyguanosine triphosphate (“ddG”), dideoxycytosine triphosphate (“ddC”), dideoxythymidine triphosphate (“ddT”), which randomly incorporates into the extending DNA polymer at different nucleotide positions, and terminates extension beyond that position. Accordingly, at the end of the process, each vessel contains a mixture of DNA polymers of different lengths, representing of all possible lengths from the point of primer extension to each nucleotide position downstream. These sets of chain-termination fragments are then separated based on molecular weight using gel electrophoresis or capillary electrophoresis techniques. Because the set of fragments represents a complete set of polymers beginning at the same nucleotide position but terminating at different downstream nucleotide positions, the molecular weight of a fragment is indicative of the terminating nucleotide position, which can be used to determine the relative order of nucleotides in the sequence.

Sequencing of polynucleotides may be performed using either single-stranded or double stranded DNA. Use of polymerase for primer extension requires a single-stranded DNA template. In preferred embodiments, the method of the present invention uses double-stranded DNA in order to obtain opposite strand confirmation of sequencing results. Double stranded DNA templates may be sequenced using either alkaline or heat denaturation to separate the two complementary DNA templates into single strands. During polymerization, each molecule of the DNA template is copied once as the complementary primer-extended strand. Use of thermostable DNA polymerases (e.g. Taq, Bst, Tth or Vent DNA polymerase) enables repeated cycling of double-stranded DNA templates in the sequencing reaction through alternate periods of heat denaturation, primer annealing, extension and dideoxy termination. This cycling process effectively amplifies small amounts of input DNA template to generate a sufficient quantity of polynucleotide template for sequencing.

Sequencing may also be performed directly on PCR amplification reaction products. Although the cloning of amplified DNA is relatively straightforward, direct sequencing of PCR products facilitates and speeds the acquisition of sequence information. As long as the PCR reaction produces a discrete amplified product, it will be amenable to direct sequencing. In contrast to methods where the PCR product is cloned and a single clone is sequenced, the approach in which the sequence of PCR products is analysed directly is generally unaffected by the comparatively high error rate of Taq DNA polymerase. Errors are likely to be stochastically distributed throughout the molecule. Thus, the majority of the amplified product will consist of the correct sequence. Direct sequencing of PCR products has the advantage over sequencing cloned PCR products in that (1) it is readily standardized because it is simple enzymatic process that does not depend on the use of living cells, and (2) only a single sequence needs to be determined for each sample.

The practice of the present invention will employ, unless otherwise indicated, conventional techniques of molecular biology, microbiology, recombinant DNA techniques, oligonucleotide synthesis which are within the skill of the art. Such techniques are explained fully in the literature. Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)); Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.), the contents of all of which are incorporated herein by reference.

### EMBODIMENTS OF THE INVENTION

The present invention is directed to methods for detecting constituent peaks in any spectral-type signal representing the relative spatial distribution of biological or chemical constituents or molecular components in a mixture, subjected to separation by chromatography or other similar methods. Methods that utilize a spectral-type signal include, but are not limited to, electrophoresis, affinity chromatography, high-pressure liquid chromatography, flow cytometry of cells and subcellular components, and the like. Further, the methods of the present invention may be also used to analyze spectral data resulting from Mass-Spectroscopy where peak widths (and hence resolution information) can be inferred based on the use of samples with known molecular weight. The methods described herein are suitable for analysis of migration patterns obtained by any of the foregoing means.

In a particular embodiment, the present invention is directed to methods for accurately analyzing data traces used in nucleic acid sequencing. Nucleic acid sequencing is a critical component of a variety of diagnostic assays, such as viral and bacterial resistance testing, genetic predisposition testing and predictive medicine testing. Sequencing-based HIV-resistance testing, for example, relies upon the use and interpretation of data traces representing the sequence of a region of HIV containing genetic mutations conferring drug resistance. Resistance of the viral strain(s), present in a patient, to specific drug regiments is inferred based on known mutations in the sequenced regions of the DNA sample. Inference of resistance to specific drugs demands accurate identification of mutations and hence the DNA sequence. The methods of the present invention, however, are generally applicable to all applications where data traces are analyzed to infer the nucleic acid composition of a sample.

The methods of the present invention can be used not only to resolve overlapping peaks, but also increase the length of sequence that can be correctly read from a given data trace referred to as read-length.

Generation of Data Trace

The present invention is directed to methods for interpreting a chromatographic data trace, which is essentially a graphical representation of physical properties of biological or chemical compounds. Because the quality of the data trace is dependent on the quality of the physical elements from which the data trace is derived, it is essential to observe standard laboratory practices relating to the procedures for generating the physical data and converting such physical data to digital or graphical form. Descriptions of common procedures used to generate a data trace are found, for example, in package inserts for typical kits used for sequencing (ABI—BigDye kit, Bayer—TRUGENE kit, etc), as well as the manuals for commercially available sequences such as MegaBACE 1000 (GE), ABI 3730 or the like.

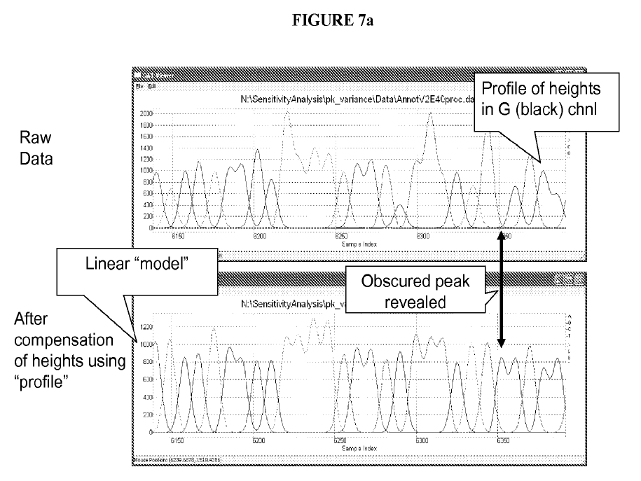

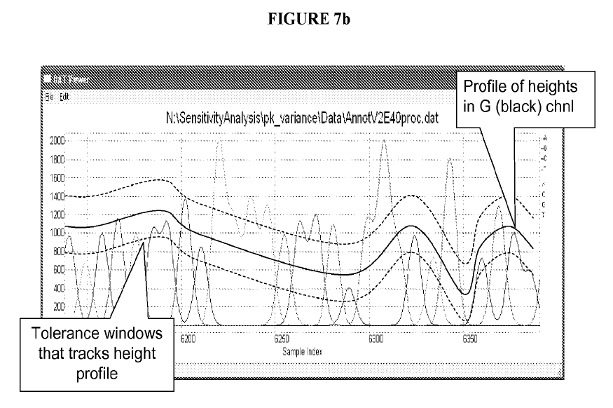

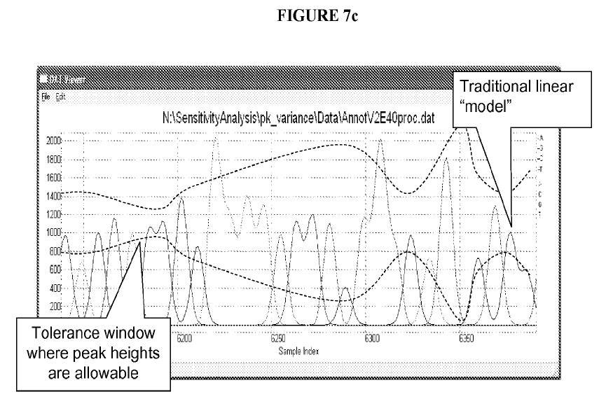

Typically, separate data traces are generated for each of four different chain-termination fragments ending with one of the standard nucleotides A, C, G, and T. These four different data traces are then combined in an initial alignment and divided into a plurality of segments or windows. FIG. 7a-c illustrates a portion of a typical data trace of a combined set of four chain-termination DNA sequencing reactions. The X-axis represents time and molecular weight, while the Y axis represents fluorescence detection. The data trace reveals a series of bands of fluorescent molecules passing through the detection site, as expected from a typical chain-termination DNA sequencing reaction.

The data traces used in the methods of the present invention are preferably a signal collected using the fluorescence detection apparatus of an automated DNA sequencer. However, the present invention is applicable to any data set which reflects the separation of oligonucleotide fragments in space or time, including real-time fragment patterns using any type of detector, for example a polarization detector as described in U.S. Pat. No. 5,543,018; densitometer traces of autoradiographs or stained gels, traces from laser-scanned gels containing fluorescently-tagged oligonucleotides: and fragment patterns from samples separated by mass spectrometry.

In addition, detector systems (such as photomultiplier, photodiode, CCD camera or autoradiographic film) should have a dynamic range of at least about two to three orders of magnitude, and the dynamic range should meet or exceed the range between the background and the most intensive bands. Additionally, care should be taken to assure that the detector is not saturated, while at the same time providing adequate detection of low-intensity bands. Third, the detector should take samples at an interval which meets the criterion of the well-known Nyquist sampling theorem. Sampling at intervals of about 0.1 to 0.5 seconds is typically sufficient, but may differ depending on the capabilities of the particular detection system and electrophoretic device. An additional criteria for the choice of sampling frequency is based on the requirement to obtain at least 5 to 6 data points per peak. Fewer data points will generally not allow accurate description of the peaks necessary to build a reliable peak model.

If the lane signal is based on a logarithmic or other nonlinear intensity scale, as is commonly true for signals produced by film scanners, it is desirable that the lane signal be linearized.

Additionally, the lane signals may be processed in digital form. Analog signals should be converted to digital lane signals before the peak resolution process is applied.

It may also be advantageous to condition the signal, although this step is not required. Signal conditioning can be done, for example, using conventional baseline correction and noise reduction techniques to yield a “conditioned” data trace. As is known in the art, three methods of signal processing commonly used are background subtraction, low frequency filtration and high frequency filtration, and any of these may be used, singly or in combination to produce a conditioned signal to be used as a conditioned data trace in the method of the invention. Preferably, the data is conditioned by background subtraction using a non-linear filter such as an erosion filter, with or without a low-pass filter to eliminate systemic noise. The preferred low-pass filtration technique is non-causal gaussian convolution.

Methods of the Invention

The present invention is generally directed to methods for detecting peaks in a data trace of a sample polynucleotide, which is capable of accounting for large variation in peak characteristics, such as peak height, by utilizing a profile of the peak characteristics generated empirically from other samples of the same polynucleotide. The methods of the present invention are particularly useful in using peak characteristics that vary stochastically, or irregularly, and do not follow a predictable pattern or trend throughout the course of the sequence signature, but which remain relatively constant, as a function of base position, from one sample of the sequence to another. For example, the methods of the present invention have been found to be particularly useful in detecting peaks in a nucleotide sequence signature using relative peak height variance as acceptance criteria. According to the classical model of peak height variation, it is assumed that the relative variation of peaks increases gradually toward the end of the sequence (i.e., peaks heights are relatively consistent at the beginning, but show significant variation toward the end of the sequence). However, peak height variance is not gradual or predictable, and cannot therefore be used as a reference standard or acceptance criteria to identify peak characteristics of constituent peaks as a means of detecting constituent peaks.

In accordance with the present invention, it has been observed that notwithstanding the stochastic nature of peak heights and peak height variance within a sequence, the characteristic of peak height variance, as a function of base position, is itself a relatively constant or conserved characteristic between different samples of the same polynucleotide. Thus, each base position of a polynucleotide sequence will have a characteristic level or value of variance, with some base positions demonstrating a high level of variance (i.e., the absolute value of peak height varies widely at that base position among different samples), while other base positions demonstrate a low level of variance (i.e., the absolute value of peak height remains relatively constant at that base position among different samples). The methods of the present invention, rather than assuming a gradual variation in peak heights, assumes that the variance or relative variance in the value of a particular peak characteristic at a given base position remains relatively constant or conserved between samples of the same polynucleotide, and utilizes such conserved characteristics from empirically observed data to generate a profile or model of acceptance criteria (i.e., the level of variation), as a function of base position, to identify constituent peaks. It is understood that the conserved characteristic may be the absolute peak height value, or alternatively may be a value representing the relative variance in peak height, relative to adjacent peaks heights or relative to a value representing the consensus or average peak height of adjacent or neighboring peaks, or both.

Peak Characteristics

The methods of the present invention utilize peak characteristics, such as a function of peak position, of reference polynucleotides to generate a profile of acceptance criteria. In another aspect of the invention, the methods utilize variation in peak characteristics, as a function of peak position, of reference polynucleotides to generate a profile of acceptance criteria. Peak characteristics may include any peak characteristic that is conserved from one sample to another, for example, peak height, peak width, peak area, or peak resolution. In a preferred aspect of the invention, a peak characteristic is any characteristic of a peak that is a function of peak height, such as peak resolution. In a more preferred aspect of the invention, the peak characteristic is peak height.

Generation of Reference Polynucleotide Sequence Signature (Profile)

Generally, the present invention involves the use of one or more, and preferably a plurality of, nucleotide sequences of a selected polynucleotide to generate a profile of peaks, as a function of peak position, which can be used as a reference standard or to generate acceptance criteria, against which the sample polynucleotide sequence is compared. The method of the present invention comprises the step of receiving a reference nucleotide sequence signature comprising signal peaks, as a function of peak position, representing a profile of one or more signal peak characteristics of a plurality of nucleotide sequence signatures of one or more reference polynucleotides. In preferred aspect of the invention, a plurality of reference polynucleotides are used to generate a profile of peak characteristics, as a function of base position. The profile is essentially a composite of empirical data, represented in the form of a reference nucleotide sequence signature. Reference polynucleotides will preferably be polynucleotides that are conserved or remain relatively constant from sample to sample. Although the reference nucleotide sequence signature is based on empirically observed peak characteristics of a plurality of actual polynucleotide sequences, which are therefore useful as a reference standard to establish criteria of the peak characteristics of a constituent peak, the reference nucleotide sequence signature itself is nevertheless a “model” that will represent composite data from a plurality of polynucleotides, and will not therefore necessarily reflect the sequence signature of any single actual polynucleotide, unless the data is obtained from only a single polynucleotide.

Reference polynucleotides may be obtained from the same source (but as a different sample) as the sample polynucleotide, or may be obtained from completely different sources. Individual reference polynucleotides obtained from various sources are then individually sequenced using nucleic acid sequencing methods well-known in the art, resulting in a plurality of nucleotide sequence signatures, one for each individual reference polynucleotide. In accordance with the present invention, the signal peaks of each of the plurality of nucleotide sequence signatures is analyzed to determine and assign a value or values for one or more particular peak characteristics, such as peak height or peak resolution, as a function of peak position. For each peak position, the peak characteristics of the various peaks at that peak position for the plurality of nucleotide sequence signatures are determined and analyzed to obtain a profile of the combined characteristics at that peak position. The profile may be expressed, for example, in the form of an average value, a mean value, a range of values, a maximum or minimum value, or a value having a predetermined deviation from a selected average, mean, maximum or minimum value. The predetermined deviation will preferably be selected by the user so as to reflect the desired limits of peak height that would be expected to represent constituent peaks at that position and exclude artifactual peaks that do not represent constituent peaks. The profile may also be expressed as values normalized by a factor sufficient to bring the peak characteristics to a unified value, with the factor used to generate a normalized profile being used to normalize the values obtained in the sample nucleotide sequence signature. By way of example, a profile of peak height at a given peak position would be generated by generating a sequence signature of a plurality of reference polynucleotides, analyzing peak heights, as a function of peak position, for all peaks in each sequence signature, and then for each peak position comparing the peak heights of the various peaks at that peak position in each of the plurality of nucleotide sequence signatures. At any given position, there will exist a set of data representing the empirically observed peak heights. The set of data are then used to generate a “profile” of the peak height data at that peak position, which may be expressed, for example, as a range of peak heights. The resulting empirically defined peak height profile thus defines criteria for detecting constituent peaks.

As discussed previously, the methods of the present invention may be carried out on a variety of computer processing devices, whether the steps are processed locally on one computer device or across multiple computing devices as a distributed system. If the later, the computer programmable code found on computer readable medium may likewise be distributed across multiple memory devices. Furthermore, the various detection systems, such as Mass-Spectroscopy devices, used to gather signals that indicate peaks in a sample nucleic acid data trace derived from a sample polynucleotide may interface with such computer system(s). Processors of the computer system(s) may be programmed to perform the steps of the methods described herein.

An input of such computer system(s) may receive information relating to a nucleic acid sequence data trace of one or more reference polynucleotides corresponding to the sample polynucleotide and receive information relating to a nucleic acid sequence data trace of a sample polynucleotide. An output of such a computer system may be provided to report detected peaks in the nucleic acid sequence data trace of the sample polynucleotide.

The methods of the present invention are directed to detecting peaks in a nucleotide sequence signature of a sample polynucleotide. In one aspect of the invention, the methods comprise a step of receiving a nucleotide sequence signature of the sample polynucleotide, wherein the sample nucleotide sequence signature comprises signal peaks, as a function of peak position, having one or more signal peak characteristics.

Thus, in one particular aspect of the present invention, the methods comprise detecting peaks in a nucleotide sequence signature of a sample polynucleotide, by utilizing a profile of peak heights generated empirically from other samples of the polynucleotide. The profile of peak heights will include, for each base position, the absolute value of peak heights observed at a given base position for one or more reference polynucleotides. If the profile of peaks heights is generated using a plurality of reference polynucleotides, the profile will reflect the range of peak heights observed for the various reference polynucleotides. A profile reflecting a range of peak heights can then be used to determine a value for empirically observed peak height variation (i.e., the value of peak height variation being the difference between the maximum peak height observed and the minimum peak height observed). Alternatively, a profile reflecting a range of peak heights can also be used to determine a value for empirically observed peak height variation (i.e., the value of peak height variation being the difference between the maximum peak height observed and the minimum peak height observed). Using this profile as the acceptance criteria for detecting a constituent peak, base positions that demonstrate wide variation in peak height among different samples can use lenient acceptance criteria commensurate with such wide variation, while base positions that demonstrate narrow variation in peak height among different samples can use stringent acceptance criteria commensurate with such wide variation the which is capable of accounting for large variation in peak heights.

Sample Polynucleotide Sequence Signature

A sample polynucleotide is a polynucleotide that is present in a sample and whose nucleotide sequence is being determined. The sample polynucleotide will be a polynucleotide that is similar or identical to a known polynucleotide. A sample polynucleotide is therefore a polynucleotide whose sequence is expected to be similar to the sequence of a conserved polynucleotide whose sequence has previously been determined from different samples. The methods of the present invention are used to accurately determine nucleotide sequences of a sample polynucleotide that has previously been sequenced and whose sequence and peaks characteristics are conserved and remain consistent from sample to sample. Where the nucleic acid sequence of a sample is known to remain relatively constant, it is possible to use sequence data (i.e., peak characteristics), obtained previously from a different sample of the same polynucleotide, as a reference standard (i.e., a “model” or “profile”) which is compared to the sequence signature of the same or similar polynucleotide obtained from a different sample. Because relative peak heights are expected to be conserved between sequencing runs of the same sample and remain fairly consistent between samples when the sequences are conserved, this prior sequence information of the same polynucleotide obtained from a different sample can be effectively used in cases where majority of the DNA sequence is known to remain constant. The present invention is therefore particularly useful in such applications as nucleic acid-based resistance testing in HIV, where approximately 90% of the viral sequence is known to remain constant from one strain of HIV to another strain obtained from a different sample. The sample polynucleotide and the reference polynucleotides are preferably homologs or orthologs, and are more preferably the same species or the same quasi-species. For example, the sample polynucleotide may be a polynucleotide obtained from any organism, including, for example, a human, a non-human animal, a bacteria, a virus, or any other organism having DNA. The different samples may be obtained from the same source as the reference polynucleotides (i.e., the same donor organism or the same tissue from the same donor organism) or may be obtained from a different source (i.e., a different donor organism, or a different tissue from the same donor organism). Where mutations are not expected in the sample sequence, the sequence signature may be generated using identical polynucleotides. Where mutations are expected in the sample sequence, the sequence signature may be generated using a consensus sequence.

Once a polynucleotide sequence signature profile is generated for the reference polynucleotides, and a polynucleotide sequence signature is generated for the sample polynucleotide, the two sequence signatures can be compared so as to correlate signal peak characteristics of each peak of the sample nucleotide sequence signature with the peak characteristics of each corresponding peak of the reference nucleotide sequence signature. The nucleotide sequence signature of the reference polynucleotide represents peak characteristics empirically determined based on one or more reference polynucleotides, which can serve as a model or profile of constituent peaks of the sample polynucleotide. Using the polynucleotide sequence signature of the reference polynucleotides as a reference standard, signal peaks of the sample sequence signature are matched or correlated with signal peaks of the reference sequence signature having similar peak characteristics. Peak characteristics of the sample sequence signature that correlate with or satisfy the criteria established by the reference sequence signature at the same base position are deemed to represent constituent peaks. By using high level contextual information present in the form of peak characteristics that are conserved at particular peak positions, the present invention is therefore able to account for variations in peak characteristics, such as peak height, provided the profile is repeatable across different runs.

The correlation of peak characteristics of the sample sequence signature with peak characteristics of the reference sequence signature may be accomplished using various alternative procedures, which may be used alone or in combination, as described in the follow sections.

Method 1—Normalized Peak Values

In one aspect of the invention, peak characteristics of the sample sequence signature are correlated with peak characteristics of the reference sequence signature by normalizing the peak characteristics of the sample sequence signature to a relatively uniform value and selecting those peaks that conform to the uniform value. In one aspect of the invention, the signal peak characteristics of the sample polynucleotide are normalized by a factor representing the inverse of the value of the peak characteristic at a corresponding position of the reference polynucleotide sequence signature. This results in peak heights that are closer to a uniform value. Hence, the data is transformed such that simple peak detection (where peak characteristics are assumed to be uniform) can be employed to identify the sequence of bases.

In one aspect of the invention, peak characteristics of the sample sequence signature are correlated with peak height of the reference sequence signature by normalizing the peak height of the sample sequence signature to a relatively uniform value and selecting those peaks that conform to the uniform value. In a particular aspect of the invention, the signal peak height of the sample polynucleotide is normalized by a factor representing the inverse of the value of the peak height at a corresponding position of the reference polynucleotide sequence signature. Hence, the data is transformed such that simple peak detection (where peak heights are assumed to be uniform) can be employed to identify the sequence of bases.

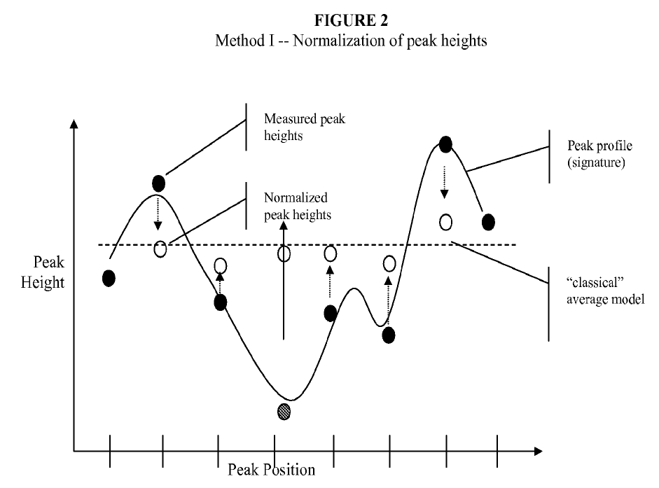

As illustrated in FIG. 2, the signal peak height of the sample polynucleotide is normalized by a factor representing the inverse of the value of the peak height at a corresponding position of the reference polynucleotide sequence signature. In reference to FIG. 2, the sinusoidal line represents signal peaks of the reference polynucleotide, with each peak having an arbitrary peak value. The sample polynucleotide sequence signature represents empirically detected and measured peaks (represented as dots), having a peak height value. The measured peaks of the sample polynucleotide sequence signature are multiplied by a factor equal to the inverse of the value of the corresponding peak height of the reference polynucleotide sequence signature, resulting in peak height values that are normalized to a relatively uniform value. Those peaks that conform to the uniform value are deemed to satisfy the criteria of a constituent peak.

The normalization process includes the following steps:

Firstly, the data trace (raw or conditioned) is searched for peaks. Peaks can be identified as the middle data point of three consecutive data points wherein the inside data point is higher than the two outside data points. More sophisticated methods of peak detection are also possible. For example, a preferred method involves using the “three-point” method to segment the data trace, and then joining the segments. A trace feature is assigned as an actual peak whenever the difference between a maximum and an adjacent minimum exceeds a threshold value, e.g., 5%. A minimum peak height from the base-line may also be required to eliminate spurious peaks.

An exception is made for the so-called “primer peak” and “termination peak” which are found in some variations of the chain-termination sequencing method. These peaks comprise a large volume of unextended primer, which tends to interfere with base-calling around the shorter chain-extension products, and a large volume of the complete sequence which may interfere with base-calling around the longest chain-extension products. These peaks are identified and eliminated from consideration either on the basis of their size, their location relative to the start and end of the electrophoresis process, or some other method.

After elimination of the primer and termination peaks, the data trace is normalized so that all of the identified peaks have the same height which is assigned a common value, e.g., 1. This process reduces signal variations due to chemistry and enzyme function, and works effectively for homozygous samples and for many heterozygotes having moderate, i.e., less than about 5 to 10%, heterozygosity in a 200 base pair or larger region being sequenced.

To normalize the data trace, the points between each peak are assigned a numerical height value based on their position in the data trace relative to a hypothetical line joining consecutive peaks and the base line of the signal. For example, if the valley between two peaks has a minimum at a point which is approximately 25% of the distance from the baseline to the line joining the peaks, then the minimum of this valley is assigned a value of about 0.25. Similarly, if the valley between two peaks has a minimum at a point which is approximately 80% of the distance from the baseline to the line joining peaks, then the minimum of this valley is assigned a value of about 0.8 in the normalized data trace.

Method 2—Absolute Peak Value

In another aspect of the invention, the profile of signal peak characteristics of the reference polynucleotides comprises a value representing a peak characteristic at each position of the reference polynucleotides. Peak characteristics of the sample sequence signature are correlated with peak characteristics of the reference sequence signature by comparing the expected values of peak characteristics of the reference polynucleotide sequence signature with the measured values of peak characteristics of the sample polynucleotide sequence signature. If the value of the signal peak characteristic of the sample polynucleotide sequence signature matches the value of the signal peak characteristic at a corresponding position of the reference polynucleotide sequence signature, then the peak is deemed to satisfy the criteria of a constituent peak.

In a particular aspect of the invention, the profile of signal peak characteristics of the reference polynucleotides comprises a value representing peak height at each position of the reference polynucleotides. Peak height of the sample sequence signature is correlated with peak height of the reference sequence signature by comparing the expected values of peak height of the reference polynucleotide sequence signature with the measured value of peak height of the sample polynucleotide sequence signature. If the value of the signal peak height of the sample polynucleotide sequence signature matches the value of the signal peak height at a corresponding position of the reference polynucleotide sequence signature, then the peak is deemed to satisfy the criteria of a constituent peak.

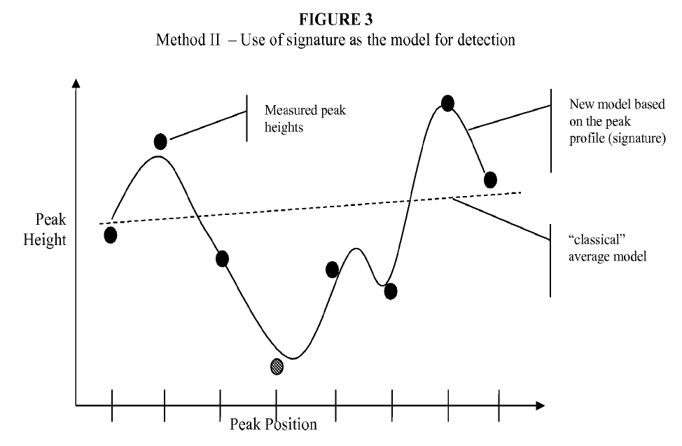

FIG. 3 illustrates the use of peak height profile as a model in peak detection. Pursuant to the classical model, peak heights are assumed to vary only gradually from the beginning of a sequence signature to the end of a sequence signature. As illustrated by the dotted line in FIG. 3, the classical model of peak height predicts that the relative variation of peaks increases gradually toward the end of the sequence (i.e., peaks heights are relatively consistent at the beginning, but show significant variation toward the end of the sequence). If such an assumption were applied, then small peaks that are comparable to background noise may not be differentiated from background noise, possibly resulting in a failure to detect the peak. In accordance with the method of the present invention, peaks that are consistently small can be captured in the peak height profile and hence when compared against this model, the peak would be a valid peak. Thus, with the method of the present invention, even small peaks can be accurately identified if the height of the particular peak at a given peak position is consistent across runs (i.e. across data obtained from multiple sequencing measurements of the same DNA fragment). Such consistency can be expressed relative to other peaks in the same chromatogram.

Method 3—Variance from Peak Values

In another aspect of the invention, the profile of signal peak characteristics of the reference polynucleotides comprises a predetermined variance from a value of a peak characteristic at each position of the reference polynucleotides. Peak characteristics of the sample sequence signature are correlated with peak characteristics of the reference sequence signature by comparing the expected values of peak characteristics of the reference polynucleotide sequence signature with the measured values of peak characteristics of the sample polynucleotide sequence signature. If the value of the signal peak characteristic of the sample polynucleotide sequence signature is within the predetermined variance of the value of the signal peak characteristic at a corresponding position of the reference polynucleotide sequence signature, then the peak is deemed to satisfy the criteria of a constituent peak.

In one aspect of the invention, the profile of signal peak characteristics of the reference polynucleotides comprises a value representing a range of acceptable peak heights at a base position. In another aspect, the profile of signal peak characteristics of the reference polynucleotides comprises a value representing a range of acceptable peak heights at a base position, as empirically observed from the reference polynucleotides. In another aspect, the profile of signal peak height of the reference polynucleotides comprises a value representing a predetermined variance at each position from a selected value. Peak height of the sample sequence signature is correlated with the range of values (or predetermined valiance from a selected peak height value) of the reference sequence signature by comparing the expected range of values (or predetermined variance from a selected value) of peak height of the reference polynucleotide sequence signature with the measured value of peak height of the sample polynucleotide sequence signature. If the value of the peak height of the sample polynucleotide sequence signature falls within the range of values (or within the predetermined variance from a selected peak height value) of the signal peak height at a corresponding position of the reference polynucleotide sequence signature, then the peak is deemed to satisfy the criteria of a constituent peak.

A peak height profile that utilizes a variance value comprises a variance from a value of a peak characteristic at a base position, the value from which the variance is calculated being based on an average value or a mean value of the peak characteristic for a set of peaks at immediately adjacent positions or for the entire sequence.

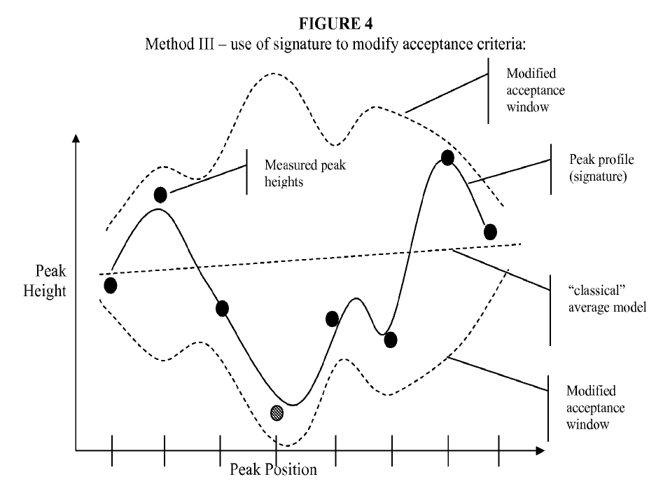

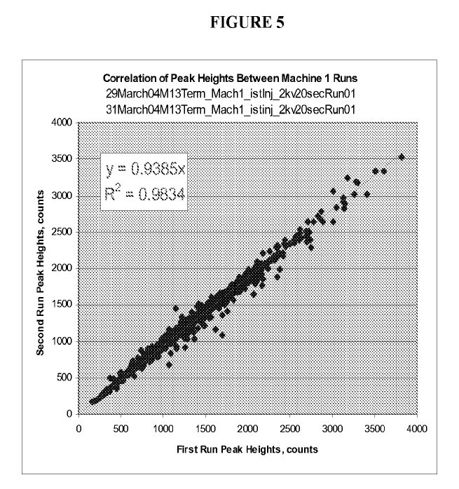

FIG. 4 illustrates the use of peak height variance as acceptance criteria for the profile. As shown by the straight dotted line in FIG. 4, the classical model of peak height predicts that the relative variation of peaks increases gradually toward the end of the sequence. If such an assumption were applied, then small peaks that are comparable to background noise (such as the one shown at the lowermost portion of the trace) may not be differentiated from background noise, possibly resulting in a failure to detect the peak. Thus, in accordance with the present invention, peaks that are consistently small can be captured in a peak height profile that defines peak height criteria at a given position in terms of a range of permissible peak height values, or in terms of a predetermined variance from a selected peak height value. In accordance with the above aspects, acceptance criteria can be independently adjusted for each base position. The modified acceptance window which is derived from the peak profile is shown by the curved dotted lines. In accordance with this approach, the peak height profile comprises acceptance criteria representing a range of values (or predetermined variance from a selected value) as a model for detecting constituent peaks. In this aspect of the invention, the classical model is still assumed to hold true; however, the window within which the peak height is allowed to vary is modified according to the peak height profile. The variation in the acceptance window, as a function of base position, ensures that small peaks are accepted when they are known to consistently occur at specific base positions. Hence, the window would be broad at positions where small peaks consistently occur (for example, the peak height designated with the shaded dot in FIG. 4) and narrow at positions where peaks are close to “classical” model (for example, the left-most peak height labeled as “measured peak heights” in FIG. 4).

The scope of the range of peak height values may be based on empirically observed peak height values of polynucleotide sequence signatures of previous sequencing runs of the same polynucleotide. Alternatively, the range of peak height values may be based on predicted peak height values, expressed as a predetermined variance from a selected peak height value, also based on the empirically observed peak height values of polynucleotide sequence signatures of previous sequencing runs of the same polynucleotide. The selected peak height value, with respect to which the predetermined variance is calculated, may be an average value of the peak heights of the reference polynucleotides at that base position, a mean value, or any other value that is useful as a point of reference from which the predetermined variance is calculated. Thus, in this aspect, even peaks that deviate significantly from the average or mean peak height values can be accurately identified if the height of the particular peak at a given peak position falls within the range of empirically observed data, or a predetermined variance therefrom.

Generation of Peak Height Profile

In accordance with the invention, the method comprises generating a profile of peak height characteristics at each base position of a polynucleotide sequence signature. This profile is generated based on the distribution of peak heights in the known sequence which is determined experimentally in one or several experiments. This may include, for example, experimental traces obtained from several capillaries in the same electrophoretic runs or in multiple runs.

### EXAMPLES

The base-calling methods of the present invention are illustrated, for example, using the well-know M13 sequence, which was experimentally registered using a capillary electrophoresis sequencer (MegaBACE 1000, GE).

FIG. 5 shows that there is a strong correlation between peak heights observed in two separate experiments (R2=0.98). This level of correlation allows use of the peak height distribution in the sequence obtained in one experiment as a signature for the second experiment. Alternatively, multiple sequences can be used to generate a single profile of peak height distribution based upon averaged values of peak height as a function of peak position.

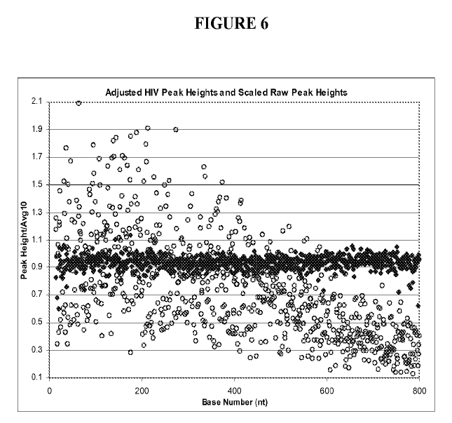

The correlation between peak heights is confirmed by the data shown in FIG. 6, which demonstrates a significant improvement in the peak height distribution after application of the sequence signature to original (raw) data. This data preprocessing allows improvement of the quality of the data used as an input into a base-calling algorithm, and as a result allows more reliable and precise base-calling.

FIGS. 7a, 7b, and 7c show sequence data traces resulting from application of the methods of the present invention to base-calling of the M13 sequence. FIG. 7a illustrates a raw data trace compared to a data trace after compensation of heights using a profile generated using a linear model in accordance with the methods of the present invention. The upper data trace in FIG. 7a (“raw data”) shows that the profile of peak heights in the raw data obscures a peak that is revealed after compensation of peak heights using a profile.

Similarly, FIG. 7b also shows that the same obscured peak shown in the upper data trace of FIG. 7a may be detected using a tolerance window that tracks the actual or empirical height profile as a function of peak position.

Finally, FIG. 7c shows that the obscured peak shown in the upper data trace of FIG. 7a may be detected using a tolerance window that reflects the value of deviation of the profile from an average value (the average value representing the traditional linear model).

While specific embodiments and applications of various methods and systems for detecting peaks in a data trace of a sample polynucleotide have been illustrated and described, it is to be understood that the invention claimed hereinafter is not limited to the precise configuration and components disclosed. Various modifications, changes, and variations apparent to those of skill in the art may be made in the arrangement, operation, and details of the methods and systems disclosed. For instance, “a processor” may include multiple processors not necessarily located in the same computer, but which carry out interrelated functions of the systems and methods described herein.

Furthermore, the methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified without departing from the scope of the invention as claimed.

The embodiments disclosed may include various steps, which may be embodied in machine-executable instructions to be executed by a general-purpose or special-purpose computer (or other electronic device). Alternatively, the steps may be performed by hardware components that contain specific logic for performing the steps, or by any combination of hardware, software, and/or firmware.

Embodiments of the present invention may also be provided as a computer program product including a machine-readable medium having stored thereon instructions that may be used to program a computer (or other electronic device) to perform processes described herein. The machine-readable medium may include, but is not limited to, floppy diskettes, optical disks, CD-ROMs, DVD-ROMs, ROMs, RAMs, EPROMs, EEPROMs, magnetic or optical cards, propagation media or other type of media/machine-readable medium suitable for storing electronic instructions. For example, instructions for performing described processes may be transferred from a remote computer (e.g., a server) to a requesting computer (e.g., a client) by way of data signals embodied in a carrier wave or other propagation medium via a communication link (e.g., network connection).

Those of skill in the art would understand that information and signals may be represented using any of a variety of different technologies and techniques. For example, data, instructions, commands, information, signals, bits, symbols, and detector and processing hardware that may be referenced throughout the above description may be represented by voltages, currents, electromagnetic waves, magnetic fields or particles, optical fields or particles, or any combination thereof.

Those of skill in the art would further appreciate that the various illustrative logical blocks, modules, processors, and algorithm steps described in connection with the embodiments disclosed herein may be implemented as electronic hardware, computer software, or combinations of both. To illustrate the interchangeability of hardware and software, various illustrative components, blocks, modules, processors, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the invention as claimed hereinafter.

1. A method for detecting peaks in a sample nucleic acid data trace derived from a sample polynucleotide, comprising:
receiving a sequence signature of a reference polynucleotide, wherein the sequence signature comprises a profile of peak height at one or more peak position of a nucleic acid sequence data trace of one or more reference polynucleotides;
receiving a sample nucleic acid sequence data trace of a sample polynucleotide corresponding to the reference polynucleotide, wherein the sample nucleic acid sequence data trace comprises a value of peak height at one or more peak position corresponding to the peak positions of the sequence signature; and
detecting peaks in the sample nucleic acid data trace having a peak height that correlates with the profile of peak height of the sequence signature at a corresponding peak position.

2. The method according to claim 1, wherein the step of detecting peaks in the sample nucleic acid data trace comprises normalizing peak heights of the sample nucleic acid sequence data trace and detecting peaks in the sample nucleic acid sequence data trace having approximately uniform height.

3. The method according to claim 2, wherein peak heights of the sample nucleic acid sequence data trace are normalized by the inverse of the peak height of a corresponding peak of the sequence signature.

4. The method according to claim 1, wherein the step of detecting peaks in the sample nucleic acid data trace comprises detecting peaks in the sample nucleic acid data trace having a peak height approximately equal to the peak height of a corresponding peak of the sequence signature.

5. The method according to claim 1, wherein the step of detecting peaks in the sample nucleic acid data trace comprises determining for each peak position of the reference polynucleotide a value encompassing the variance in peak height from an average peak height of a plurality of nucleic acid bases of the reference polynucleotide, and detecting peaks in the sample nucleic acid data trace at a corresponding base position having a peak height within said variance.

6. The method according to claim 1, wherein the sequence signature of the reference polynucleotide comprises a profile of peak height of peaks of one or more nucleic acid bases as a function of peak position of a nucleic acid sequence data trace of a single reference polynucleotide.

7. The method according to claim 6, wherein the step of detecting peaks in the sample nucleic acid data trace comprises normalizing peak heights of the sample nucleic acid sequence data trace and detecting peaks in the sample nucleic acid sequence data trace having approximately uniform height.

8. The method according to claim 7, wherein peak heights of the sample nucleic acid sequence data trace are normalized by the inverse of the peak height of a corresponding peak of the sequence signature.

9. The method according to claim 6, wherein the step of detecting peaks in the sample nucleic acid data trace comprises detecting peaks in the sample nucleic acid data trace having a peak height approximately equal to the peak height of a corresponding peak of the sequence signature.

10. The method according to claim 6, wherein the step of detecting peaks in the sample nucleic acid data trace comprises determining for each peak position of the reference polynucleotide a value encompassing the variance in peak height from an average peak height of a plurality of nucleic acid bases of the reference polynucleotide, and detecting peaks in the sample nucleic acid data trace at a corresponding base position having a peak height within said variance.

11. An apparatus for detecting peaks in a sample nucleic acid data trace derived from a sample polynucleotide, comprising:
(a) an input to receive information relating to a nucleic acid sequence data trace of one or more reference polynucleotides corresponding to the sample polynucleotide and to receive information relating to a nucleic acid sequence data trace of a sample polynucleotide;
(b) one or more processor operatively programmed to collectively perform the following processes:
evaluate the nucleic acid sequence data trace of the one or more reference polynucleotides and generate a sequence signature comprising a profile of peak height, as a function of peak position, at one or more peak position of the nucleic acid sequence data trace of the one or more reference polynucleotide;
evaluate the sample nucleic acid sequence data trace and generate a value of peak height, as a function of peak position, at one or more peak position corresponding to a peak position of the sequence signature;
detect peaks in the sample nucleic acid data trace having a peak height that correlates with the profile of peak height of the sequence signature at a corresponding peak position, thereby identifying valid peaks in the nucleic acid sequence data trace of the sample polynucleotide; and
(c) an output to report detected peaks in the nucleic acid sequence data trace of the sample polynucleotide.

12. The apparatus of claim 11, wherein the processor is programmed to detect peaks in the sample nucleic acid data trace by normalizing peak heights of the sample nucleic acid sequence data trace and to detect peaks in the sample nucleic acid sequence data trace having approximately uniform height.

13. The apparatus of claim 12, wherein peak heights of the sample nucleic acid sequence data trace are normalized by the inverse of the peak height of a corresponding peak of the sequence signature.

14. The apparatus of claim 11, wherein the processor is programmed to detect peaks in the sample nucleic acid data trace having a peak height approximately equal to the peak height of a corresponding peak of the sequence signature.

15. The apparatus of claim 11, wherein the processor is programmed to determine, for each peak position of the reference polynucleotide, a value encompassing the variance in peak height from an average peak height of a plurality of nucleic acid bases of the reference polynucleotide, and to detect peaks in the sample nucleic acid data trace at a corresponding base position having a peak height within said variance.

16. A computer system for detecting peaks in a sample nucleic acid data trace derived from a sample polynucleotide, comprising:
(a) an input to receive information relating to a nucleic acid sequence data trace of a reference polynucleotide corresponding to the sample polynucleotide and to receive information relating to a nucleic acid sequence data trace of a sample polynucleotide;
(b) one or more processor operatively programmed to collectively perform the following processes:
evaluate the nucleic acid sequence data trace of the reference polynucleotide and generate a sequence signature comprising a profile of peak height, as a function of peak position, at one or more peak position of the nucleic acid sequence data trace of the reference polynucleotide;
evaluate the sample nucleic acid sequence data trace and generate a value of peak height, as a function of peak position, at one or more peak position corresponding to a peak position of the sequence signature;
detect peaks in the sample nucleic acid data trace having a peak height that correlates with the profile of peak height of the sequence signature at a corresponding peak position, thereby identifying valid peaks in the nucleic acid sequence data trace of the sample polynucleotide; and
(c) an output to report detected peaks in the nucleic acid sequence data trace of the sample polynucleotide.

17. The computer system of claim 16, wherein the processor is programmed to detect peaks in the sample nucleic acid data trace by normalizing peak heights of the sample nucleic acid sequence data trace and detecting peaks in the sample nucleic acid sequence data trace having approximately uniform height.

18. The computer system of claim 17, wherein peak heights of the sample nucleic acid sequence data trace are normalized by the inverse of the peak height of a corresponding peak of the sequence signature.

19. The computer system of claim 16, wherein the processor is programmed to detect peaks in the sample nucleic acid data trace having a peak height approximately equal to the peak height of a corresponding peak of the sequence signature.

20. The computer system of claim 16, wherein the processor is programmed to determine, for each peak position of the reference polynucleotide, a value encompassing the variance in peak height from an average peak height of a plurality of nucleic acid bases of the reference polynucleotide, and to detect peaks in the sample nucleic acid data trace at a corresponding base position having a peak height within said variance.

21. A computer readable medium having stored thereon computer executable instructions for performing a method for detecting peaks in a sample nucleic acid data trace derived from a sample polynucleotide, the method comprising:
receiving a sequence signature of a reference polynucleotide, wherein the sequence signature comprises a profile of peak height at one or more peak position of a nucleic acid sequence data trace of one or more reference polynucleotides;
receiving a sample nucleic acid sequence data trace of a sample polynucleotide corresponding to the reference polynucleotide, wherein the sample nucleic acid sequence data trace comprises a value of peak height at one or more peak position corresponding to the peak positions of the sequence signature; and
detecting peaks in the sample nucleic acid data trace having a peak height that correlates with the profile of peak height of the sequence signature at a corresponding peak position.

22. The computer readable medium according to claim 21, wherein the step of detecting peaks in the sample nucleic acid data trace comprises normalizing peak heights of the sample nucleic acid sequence data trace and detecting peaks in the sample nucleic acid sequence data trace having approximately uniform height.

23. The computer readable medium according to claim 22, wherein peak heights of the sample nucleic acid sequence data trace are normalized by the inverse of the peak height of a corresponding peak of the sequence signature.

24. The computer readable medium according to claim 21, wherein the step of detecting peaks in the sample nucleic acid data trace comprises detecting peaks in the sample nucleic acid data trace having a peak height approximately equal to the peak height of a corresponding peak of the sequence signature.

25. The computer readable medium according to claim 21, wherein the step of detecting peaks in the sample nucleic acid data trace comprises determining for each peak position of the reference polynucleotide a value encompassing the variance in peak height from an average peak height of a plurality of nucleic acid bases of the reference polynucleotide, and detecting peaks in the sample nucleic acid data trace at a corresponding base position having a peak height within said variance.

26. The computer readable medium according to claim 21, wherein the sequence signature of the reference polynucleotide comprises a profile of peak height of peaks of one or more nucleic acid bases as a function of peak position of a nucleic acid sequence data trace of a single reference polynucleotide.

27. The computer readable medium according to claim 26, wherein the step of detecting peaks in the sample nucleic acid data trace comprises normalizing peak heights of the sample nucleic acid sequence data trace and detecting peaks in the sample nucleic acid sequence data trace having approximately uniform height.

28. The computer readable medium according to claim 27, wherein peak heights of the sample nucleic acid sequence data trace are normalized by the inverse of the peak height of a corresponding peak of the sequence signature.

29. The computer readable medium according to claim 26, wherein the step of detecting peaks in the sample nucleic acid data trace comprises detecting peaks in the sample nucleic acid data trace having a peak height approximately equal to the peak height of a corresponding peak of the sequence signature.

30. The computer readable medium according to claim 26, wherein the step of detecting peaks in the sample nucleic acid data trace comprises determining for each peak position of the reference polynucleotide a value encompassing the variance in peak height from an average peak height of a plurality of nucleic acid bases of the reference polynucleotide, and detecting peaks in the sample nucleic acid data trace at a corresponding base position having a peak height within said variance.
